(19) United States
(12) Patent Application Publication
(10) Pub. No.: US 2013/0310771 A1
(43) Pub. Date: Nov. 21, 2013

(54) MEDICAL CONDUIT PROTECTION DEVICES, SYSTEMS AND METHODS

(71) Applicant: Daniel Karlin, Medford, MA (US)

(72) Inventor: Daniel Karlin, Medford, MA (US)

(21) Appl. No.: 13/894,598

(22) Filed: May 15, 2013

Related U.S. Application Data
(60) Provisional application No. 61/647,388, filed on May 15, 2012.

(51) Int. Cl. A61M 25/02

(52) U.S. Cl. CPC A61M 25/02
USPC 604/263

(57) ABSTRACT

A medical conduit protection device has, for example, an elongate body having a wall defining a lumen for one or more elongate medical conduits. The device also includes first and second tethers provided on opposed ends of the body for securing the device to a point on or near a patient, and an external point. The medical conduit protection device inhibits tampering with or damage to the one or more medical conduits within the lumen of the body by shielding, gathering, transferring externally-applied strain away from, or otherwise protecting the one or more medical conduits within the lumen thereof.

### CROSS-REFERENCE TO RELATED APPLICATION

This patent application claims the benefit of priority to U.S. Provisional Patent Application Ser. No. 61/647,388, filed May 15, 2012, which is incorporated by reference herein in its entirety.

### BACKGROUND OF THE INVENTION

1. Field of the Invention

The subject invention is directed to the field of medical devices, and more particularly to devices for use in protecting patient-connected structures such as tubes and wires, including but not limited to IV tube sets, catheters and sensor wires.

2. Description of Related Art

Various patient-connected structures find use in medical facilities, in operating rooms, patient rooms, and the like. Such structures include, for example, intravenous (I.V.) infusion sets, electrical leads for electrocardiogram (EKG), electroencephalogram (EEG), oxygen sensors, wound drains, suction tubing, urinary catheter tubing, feeding tubes and still other structures.

While placement of such structures is necessary to treat injuries or symptoms, to alleviate pain and discomfort that a patient is enduring and, in many cases, keep the patient alive, such structures and the connections thereof are susceptible to tampering and damage, particularly in cases of uncooperative patients, and more particularly in cases of mentally-ill patients.

Applicant recognizes that it would be advantageous to provide devices in a clinical setting that could be employed to protect the aforementioned structures from tampering, damage and improper removal, thereby protecting the patient's wellbeing to the extent possible.

### SUMMARY OF THE PREFERRED EMBODIMENTS

In accordance with one aspect of the present invention, a medical conduit protection device is provided having an elongate body having a wall, defining a lumen therein, and having first and second opposed ends, the body being adapted and configured to receive, within the lumen thereof, one or more elongate medical conduits. The device also includes a first tether provided on the first end of the body, the first tether being adapted and configured to secure the first end of the body to a patient-end anchor point (such as a patient or a location relatively near to a patient, such as a bed rail) and a second tether provided on the second end of the body, the second tether being adapted and configured to secure the second end of the body to an external anchor point. The medical conduit protection device inhibits tampering with or damage to the one or more medical conduits within the lumen of the body by transferring externally-applied strain away from the one or more medical conduits within the lumen thereof.

The body can be a substantially continuous tubular structure, adapted and configured to receive one or more medical conduits therethrough, from the first end to the second end thereof. The body can be formed from a resilient material which is resistant to plastic deformation. Alternatively, the body can be formed from a nonwoven fabric. The body can be provided in any needed dimensions practicable, such as with a diameter of between about 2.0 centimeters and about 5.0 centimeters, for example.

In accordance with a further aspect of the invention, the subject medical conduit protection devices can include one or more closure elements adapted and configured for inhibiting access to the one or more medical conduits within the lumen thereof.

In accordance with the invention, the subject medical conduit protection devices can include at least one closure element provided in connection with the body, for inhibiting access to the lumen thereof. The at least one closure element can be a reversibly attached closure element. Further, the at least one closure element can be adapted and configured to require a separate tool for removal thereof. The at least one closure element can be a one-way closure element. The at least one closure element can be a concentrically rotatable tubular element arranged over the body.

In accordance with a further aspect of the invention, the body can include at least one inflatable element, the at least one inflatable element being adapted and configured to impart structural rigidity to at least a portion of the body.

Additionally or alternatively, the body can include at least one semi-rigid element, the at least one semi rigid element being adapted and configured to impart structural rigidity to at least a portion of the body. The at least one semi-rigid element can be a helical spine provided in connection with the wall of the body.

In accordance with the invention, the subject medical conduit protection devices can include an alarm adapted and configured to sound when detecting tampering with the medical conduit protection device.

In accordance with the invention, the subject medical conduit protection devices can additionally or alternatively include a safety release feature, adapted and configured to release when a force exceeding a predetermined value is applied to the medical conduit protection device. An alarm can be provided in connection with the safety release feature, adapted and configured such that when the force exceeding the predetermined value is applied to the medical conduit protection device, the alarm is sounded.

The safety release features can include, but are not limited to one or more lines of weakness, severable connection, releasable connection and/or elastic elements.

In accordance with a further aspect of the invention, a method of protecting medical conduits from damage or tampering is provided, including the steps of providing a medical conduit protection device, the medical conduit protection device being adapted and configured for attachment at a first end to a patient-end anchor point and at a second end to an external anchor point apart from the patient, arranging the medical conduit protection device about one or more medical conduits so as to inhibit access to the one or more medical conduits, and to distribute externally-applied forces away from the one or more medical conduits, securing the first end of the protection device to the patient-end anchor point, securing the second end of the protection device to an anchor point apart from the patient, and connecting the one or more medical conduits at the first end to the patient or a location near the patient and at the second end to medical equipment.

In accordance with still a further aspect of the invention, a medical conduit protection device is provided, having an elongate body having a wall, defining a lumen therein, and having first and second opposed ends, the body being adapted and configured to receive, within the lumen thereof, one or more elongate medical conduits, the first end of the body being adapted and configured to be secured directly to a patient-end anchor point, the medical conduit protection device inhibiting tampering with or damage to the one or more medical conduits within the lumen of the body by shielding from damage the one or more medical conduits within the lumen thereof.

### BRIEF DESCRIPTION OF THE DRAWINGS

So that those skilled in the art to which the subject invention appertains will readily understand how to make and use the medical conduit protection devices, systems and related methods of the subject invention without undue experimentation, exemplary embodiments thereof are described in detail below along with the related methods, with reference to the drawings, wherein:

FIG. 1 is a schematic side view of a medical conduit protection device in accordance with a first embodiment of the subject invention, which includes a tether at each opposed end, and one or more closure elements disposed along the body thereof, only one of which being illustrated for simplicity;

FIG. 2 is a cross-sectional view of the medical conduit protection device of FIG. 1, taken along line A-A of FIG. 1;

FIG. 3 is a side view of a medical conduit protection device in accordance with a second embodiment of the invention, wherein a patient-end tether is provided as an adjustable cuff;

FIG. 4 is a detail view of a patient-end tether of the medical conduit protection device of FIG. 3;

FIG. 5 is a detail view of a far end of the medical conduit protection device of FIG. 3, illustrating a longitudinal slit in the body thereof;

FIG. 6 is a detail view of a far end of the medical conduit protection device of FIG. 3, illustrating one example of a tether for connection to an external anchor point;

FIG. 7 is a detail view of a patient-end tether of the medical conduit protection device of FIG. 3, as applied to a patient's arm;

FIG. 8 is a detail view of the medical conduit protection device of FIG. 3, further illustrating a longitudinal slit in the body thereof;

FIG. 9 is a side view of a medical conduit protection device in accordance with a third embodiment of the invention, wherein a relatively large diameter body is provided, in conjunction with one more closures arranged circumferentially about the body in one or more locations along the axis of the body;

FIG. 10 is a side view of a medical conduit protection device in accordance with a fourth embodiment of the invention, wherein rotatable closure elements are provided for inhibiting access to conduits held within a lumen of the device;

FIG. 11 is a cross sectional view of the medical conduit protection device of FIG. 10, taken across line B-B of FIG. 10;

FIG. 12 is an isometric view of a medical conduit protection device in accordance with a fifth embodiment of the invention, wherein a helical structural element is provided in connection with the body of the device;

FIG. 13 is a cross sectional view of the medical conduit protection device of FIG. 12, taken across line C-C of FIG. 12;

FIG. 14 is an isometric view of a medical conduit protection device in accordance with a sixth embodiment of the invention, wherein inflatable channels are provided in connection with the body of the device in a longitudinal and circumferential pattern to enhance structural rigidity thereof;

FIG. 15 is an isometric view of a medical conduit protection device in accordance with a seventh embodiment of the invention, wherein inflatable channels are provided in connection with the body of the device in a diagonal pattern to enhance structural rigidity thereof;

FIG. 16 is a partial side isometric view of a medical conduit protection device in accordance with an eighth embodiment of the invention, which is provided with a safety release feature, in which connections between two segments of a body are connected by severable or otherwise releasable connections;

FIG. 17 is a partial side isometric view of a medical conduit protection device in accordance with a ninth embodiment of the invention, which is provided with a safety release feature, in which connections between two segments of a continuous body are folded over one another and connected by severable or otherwise releasable connections;

FIG. 18 is a partial side isometric view of a medical conduit protection device in accordance with an tenth embodiment of the invention, which is provided with a safety feature, in which a segment of a body is formed of a resilient material to permit limited stretching of the body of the device;

FIG. 19 is a partial side isometric view of a medical conduit protection device in accordance with an eleventh embodiment of the invention, which is provided with a safety release feature, in which a line of weakness is provided in the body;

FIG. 20 is a side view of a medical conduit protection device in accordance with a twelfth embodiment of the invention, which is provided with an alarm to dissuade tampering with the device;

FIG. 21 illustrates a medical conduit protection device in accordance with the invention in use, secured between an IV pole and a bed rail;

FIG. 22 is a side view of a medical conduit protection device in accordance with a thirteenth embodiment of the invention, wherein attached tethers are not provided at the ends thereof; and

FIG. 23 is an isometric view of a medical conduit protection device in accordance with a thirteenth embodiment of the invention in which a lumen of the body of the device is contiguous with a lumen formed in a cuff of a patient-end tether thereof, and in which a portion of the cuff is made from a transparent material to permit monitoring of an insertion site of patient-connected medical conduits.

### DETAILED DESCRIPTION

The present invention relates to a medical tubing and wire (in general, “medical conduits”) protection devices, and the related systems and methods. As used herein, the term “medical conduits” can include, but should not be construed as being limited to intravenous tubing, other tubing such as drains and catheters, wires or other patient-connected structures. Moreover, Applicant specifically conceives that the subject devices can advantageously be applied to protect structures or devices which may not necessarily be “conduits.” Nevertheless, the term “medical conduit(s)” is used herein for simplicity.

In a clinical setting, patients who are unable or unwilling to cooperate and adhere to recommendations regarding limiting movement and not disrupting medical tubes or wires are often encountered. Such patients, for example, may include pediatric, demented, delirious, psychiatric or otherwise uncooperative patients. In such patients, maintaining the patency of the various medical tubes and wires may prove difficult as such patients often attempt to tamper with or remove IV-lines, catheters or sensors. Such behavior, naturally, can jeopardize such patients' wellness.

The subject medical conduit protection devices, example embodiments of which are discussed herein and illustrated in the appended figures, are configured to protect patient-attached medical conduits. It should be noted, however, that devices in keeping with the invention could be applied in still other situations, in which protection of structures is desired, which are not necessarily attached to, near, or for the benefit of a patient. Moreover, the subject devices may advantageously find use in a veterinary setting. In patients who might be expected to present the aforementioned difficulties or already have proven difficult, the device can be placed around vulnerable tubes and wires and the each end secured in such a way that forces, which would otherwise be placed on these tubes and wires (conduits), would be transferred to the protection device instead. So, for example, one end of the device can be attached to a patient's limb, with the IV catheter inserted in it, and with the other end of the device secured to an IV pole, for example. Such an arrangement advantageously allows the IV line to pass as it normally would, with its function unaffected, while any movement of the limb that would otherwise tension the conduits and risk violation thereof is be transferred to the protection device instead. Furthermore, depending on the precise implementation, the area of the conduits that would be vulnerable to kinking, bending, cutting, or biting by the patient would be covered by the protective surface of the device and thus protected.

Devices in accordance with the invention, in general, support medical conduits and protect such conduits from tampering and damage. In one aspect, devices in accordance with the invention substantially or completely surround one or more medical conduits, and thus prevent damage to these tubes from longitudinal or transverse pulling, intentional kinking, assaults with hands or sharp objects, or biting, for example. Advantageously, in collecting a plurality of medical conduits within one device can also serve to enhance safety in a clinical environment my reducing the usual array of patient-attached structures to a single device when practicable, simplifying the environment surrounding the patient, thereby reducing the tripping and/or entanglement hazard that multiple individual conduits often create.

In accordance with one aspect of the invention, it is preferred that a body of the subject devices be formed from a material that resists cutting, kinking, biting, and other physical assaults. Depending on the implementation, the body of the subject devices can be relatively close to that of the contained conduits, closely bundling such conduits. In such embodiments, as well as in other embodiments described herein, a longitudinal opening (e.g. slit) can be provided to permit transverse insertion of medical conduits. Such an arrangement is advantageous in that the conduits can be gathered and collected within subject protection devices while the conduits are already attached to the patient, while additional conduits can be easily added to the others at a later time. Alternatively, the body of the subject devices can be dimensioned to be substantially larger than that of the contained conduits, which can facilitate insertion of the medical conduits from an end, along the length of the body, if desired. Such an arrangement advantageously reduces potential for tampering, as there are no closures that would be susceptible to damage.

In accordance with the invention, the subject devices can be provided in any practicable dimensions. However, it is believed that protection devices having an inner diameter, that is, of a lumen running the length of the protection devices, of between about 2.0 and 5.0 centimeters. and a length of between about 0.5 and 2.0 meters. The length of the subject protection devices, in general, should be sufficient to at least protect a vulnerable length of the medical conduits, typically a section of such conduits nearest the patient, and need not necessarily extend a majority of the length of the medical conduits.

Further, as will be appreciated by the reader, the subject devices, depending on their precise configuration, can be deployed in any of a variety of lengths, for example in embodiments that are provided with a relatively soft wall, the wall can be gathered at one or both ends thereof, thus reducing the overall effective length of such devices, as needed.

The subject protection devices are preferably further provided with connections or tethers at each end thereof for securing the protection devices in a stable and safe manner. For example, a patient-end tether can be configured as a soft cuff, for the safety and comfort of the patient, while a tether at a far end can be a simple tie, such as a lace or a cord for example.

The body and tethers of the subject protection devices are preferably provided with sufficient strength so as to be able to effectively resist tension due to longitudinal pulling or transverse application of force. In accordance with a further aspect of the invention, however, the subject devices can be provided with one or more safety features that yield at a predetermined level of force, releasing the device at an end or along the body thereof. In such implementations, incorporation of an alarm feature, which would sound in case of application of excessive force, can be included.

The subject protection devices can be configured such that a natural or relaxed state of the material from which it is made automatically surrounds the medical conduits to be protected. For example if the body is formed from a resilient tubular material having a single lengthwise slit formed therein, the body can be deformed to reveal the lumen and permit lateral insertion of medical conduits, the material then relaxing into a closed configuration around the conduits once the deforming force is removed. Alternatively or additionally, one or more closure elements can be provided in connection with the body of the subject devices. Such closure elements can be of any suitable configuration, including but not limited to lace ties, straps, belts-and-buckles, hook-and-loop fasteners, adhesive tape, one or more zippers such as a lengthwise zipper, for example, an interlocking dovetail, a continuous press-to-seal closure (akin to those applied to some plastic sandwich bags), interlocking teeth or otherwise mechanically interlocking features and so on. Such features, it should be noted, can be provided at discrete positions along a length of the devices, and/or as a continuous closure along the length of the devices.

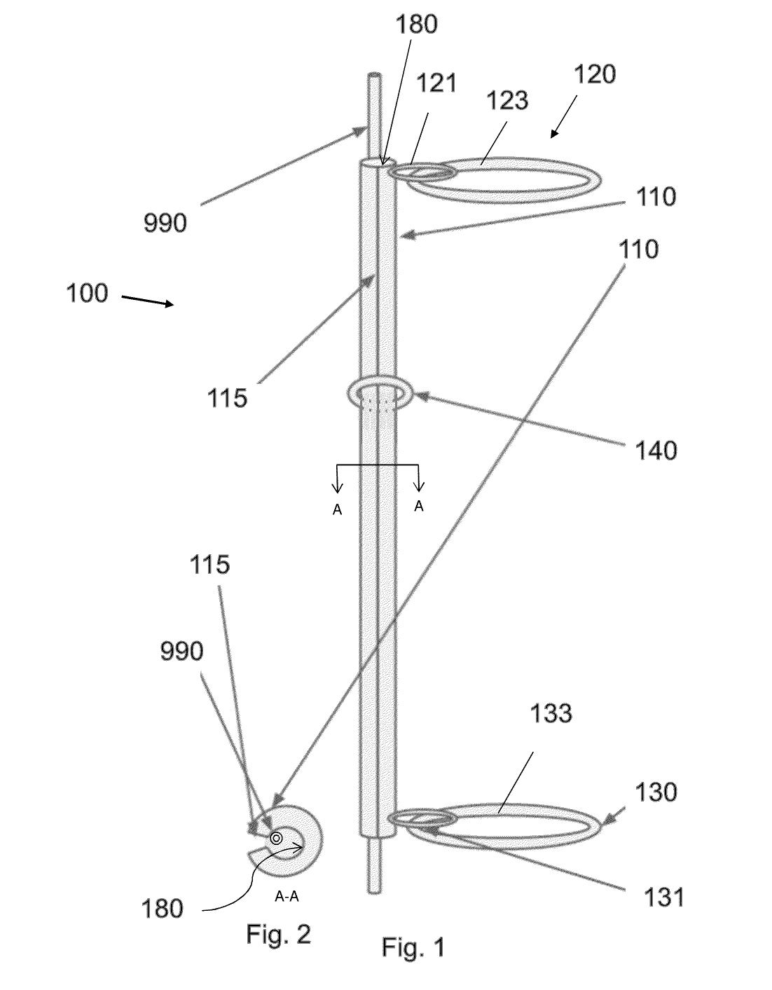

Turning now to the drawings, there is illustrated in FIG. 1, a schematic side view of a medical conduit protection device 100 in accordance with a first embodiment of the subject invention, which includes tethers 120, 130 at each opposed end. As illustrated, a first tether 120 includes a cuff portion 123 and a connection 121 to the body 110, and the second tether 130 includes a strap portion 133 and a connection 131 to the body 110. It is to be noted however, that tethers in accordance with the invention, can be secured directly to the body by any suitable construction method including, but not limited to, stitching, heat, solvent or ultrasonic welding, or other methods, depending on the materials used.

The one or more closure elements 140 are disposed along the body 110 thereof, which body 110 includes a longitudinal slit 115 that permits access to the lumen 180 defined therein. As best illustrated in the cross-sectional view of FIG. 2, medical conduits 1990 can be arranged within the lumen 180.

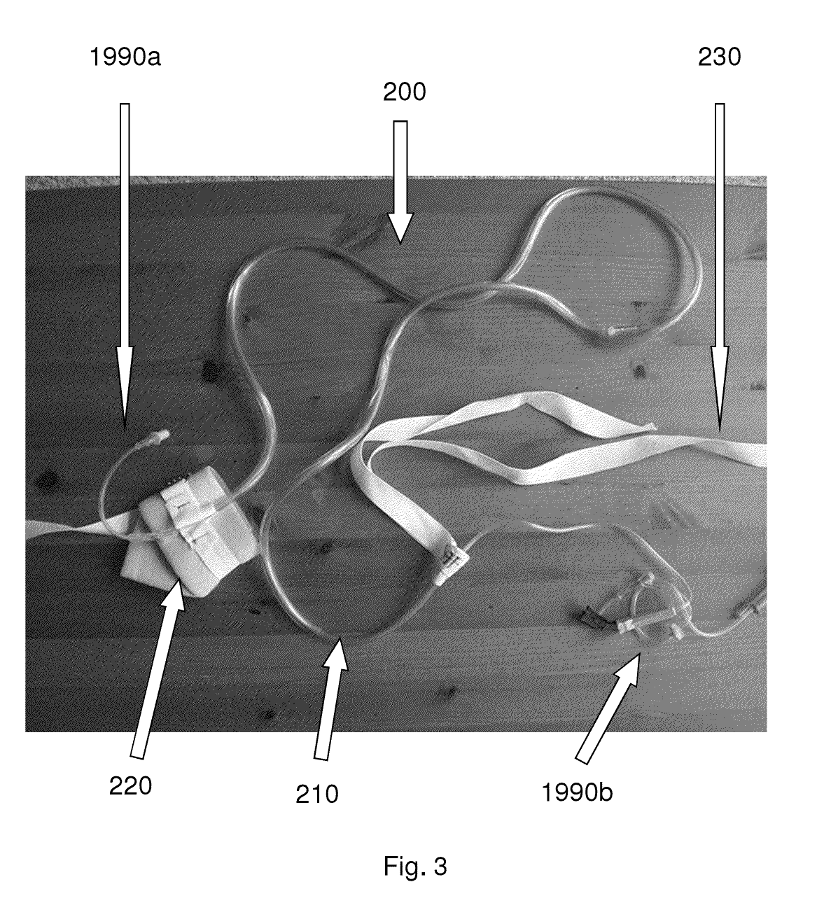

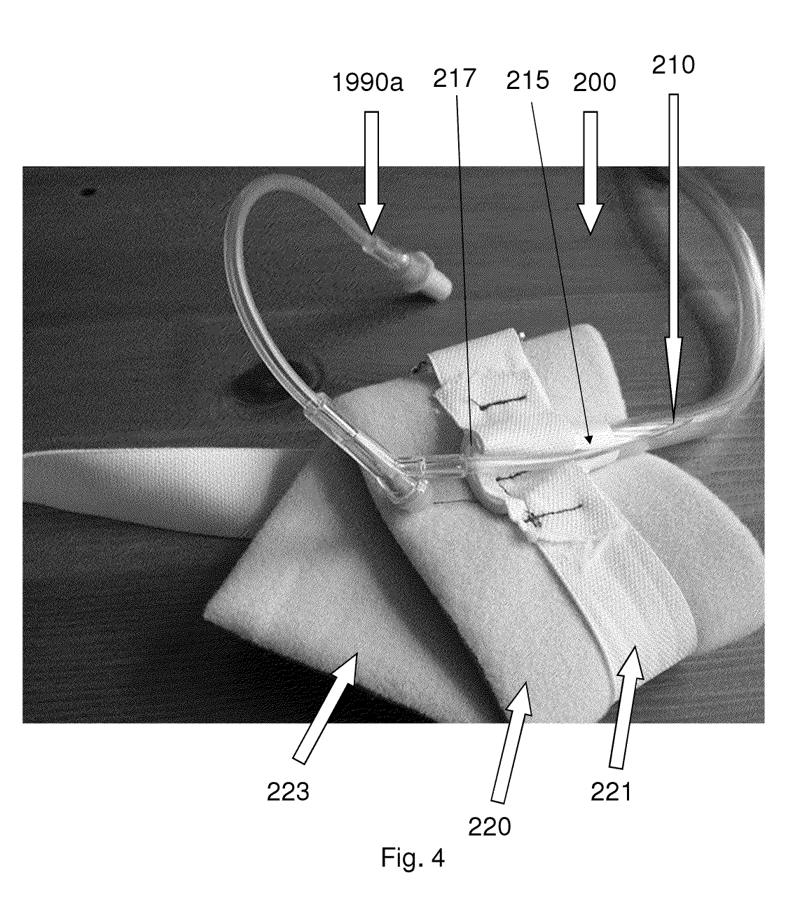

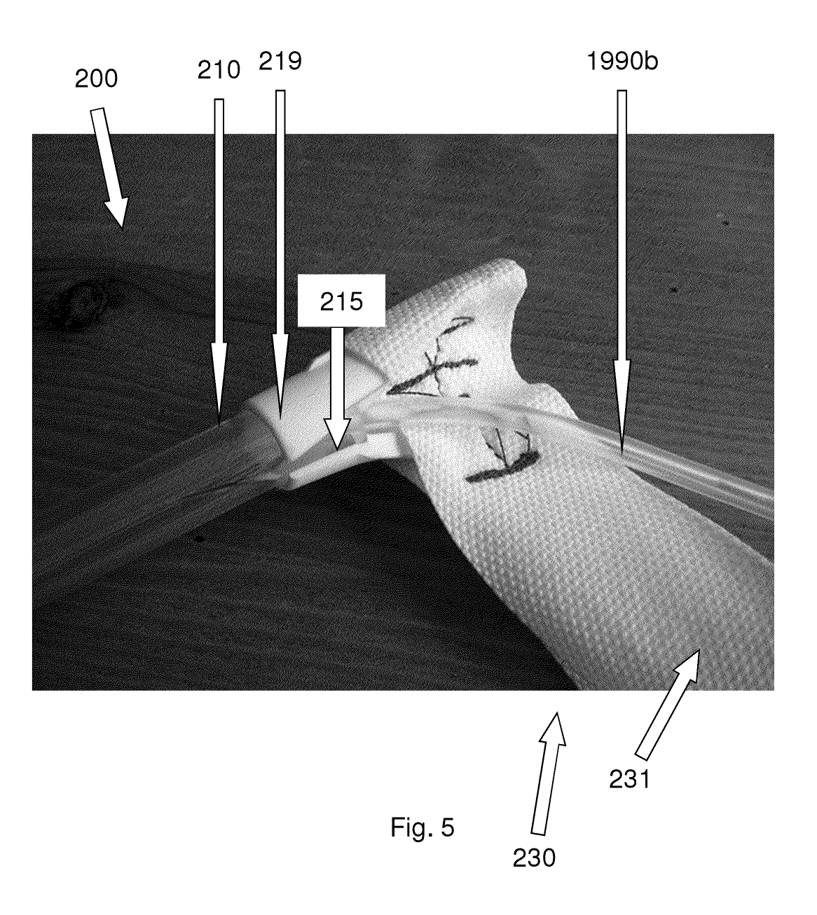

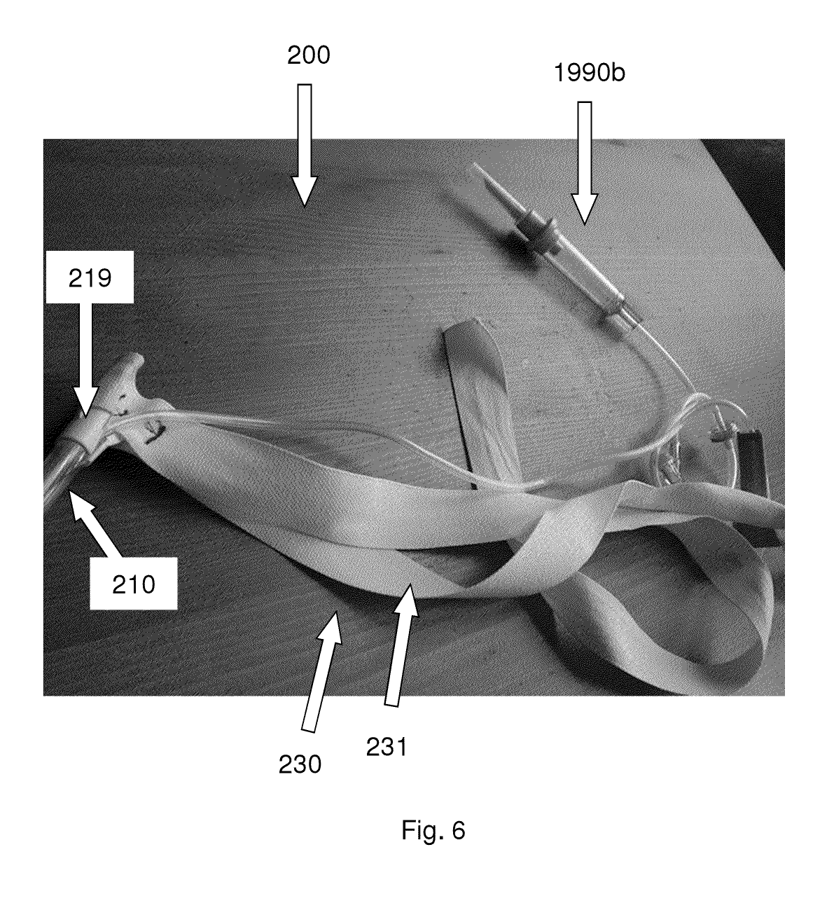

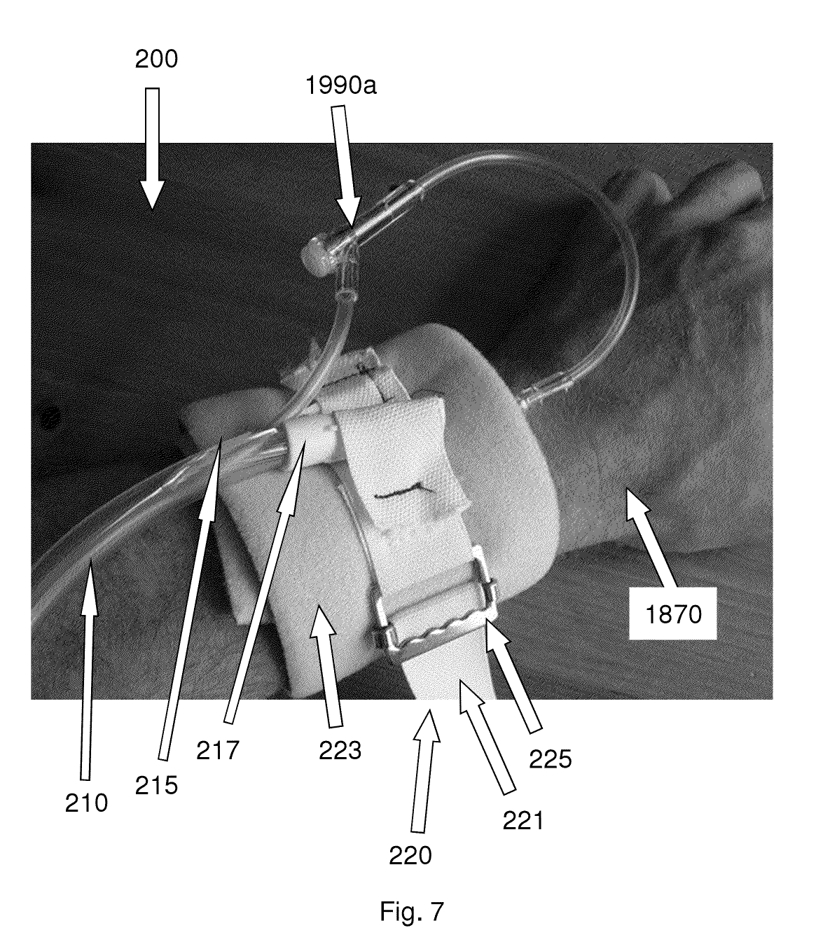

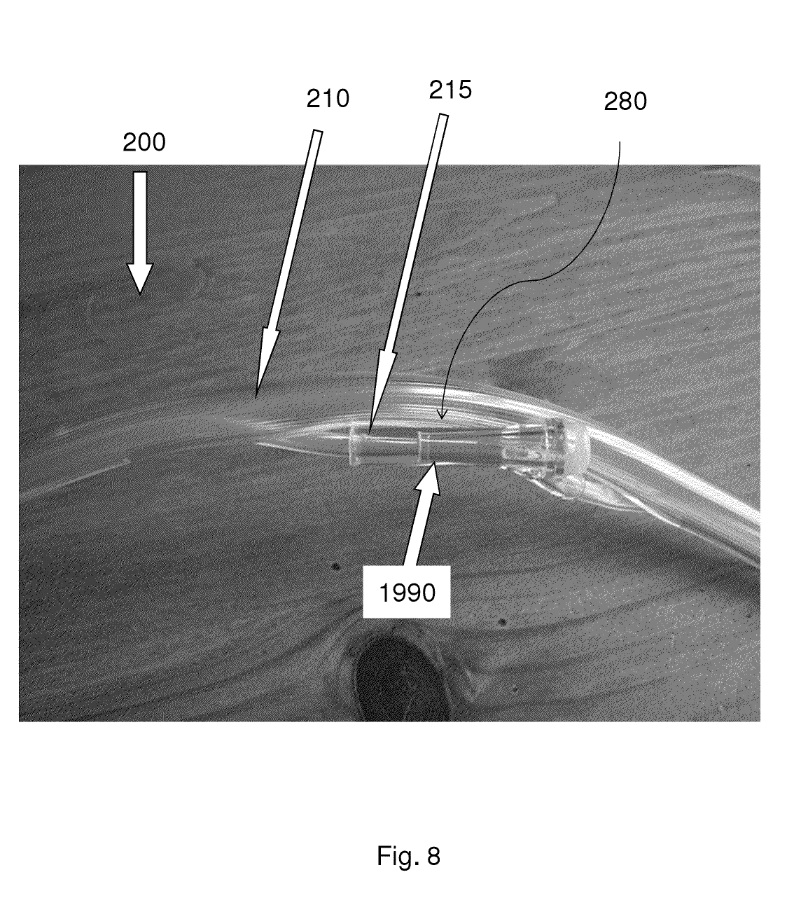

FIGS. 3-8 illustrate a medical conduit protection device 200 in accordance with a second embodiment of the invention, wherein a first or patient-end tether 220 is provided as an adjustable cuff 223. As best seen in FIGS. 4 and 7, the first tether to 220 also includes a closure having a belt 221 and a buckle 225 for safe and comfortable securing to a patient's arm 1870. As can be seen in FIG. 4, the slit 215 of the body 210 permits reception of one or more medical conduits 1990a, the medical conduits continuing out of the lumen 280 at a first end 217 thereof. Similarly, as shown in FIG. 5, the medical conduits 1990a also exit the body tube 210 at the opposite, second end 219 thereof. As seen, for example, in FIG. 6, the second tether 230 includes a pair of lace ties for securing to an external anchor point such as an IV pole.

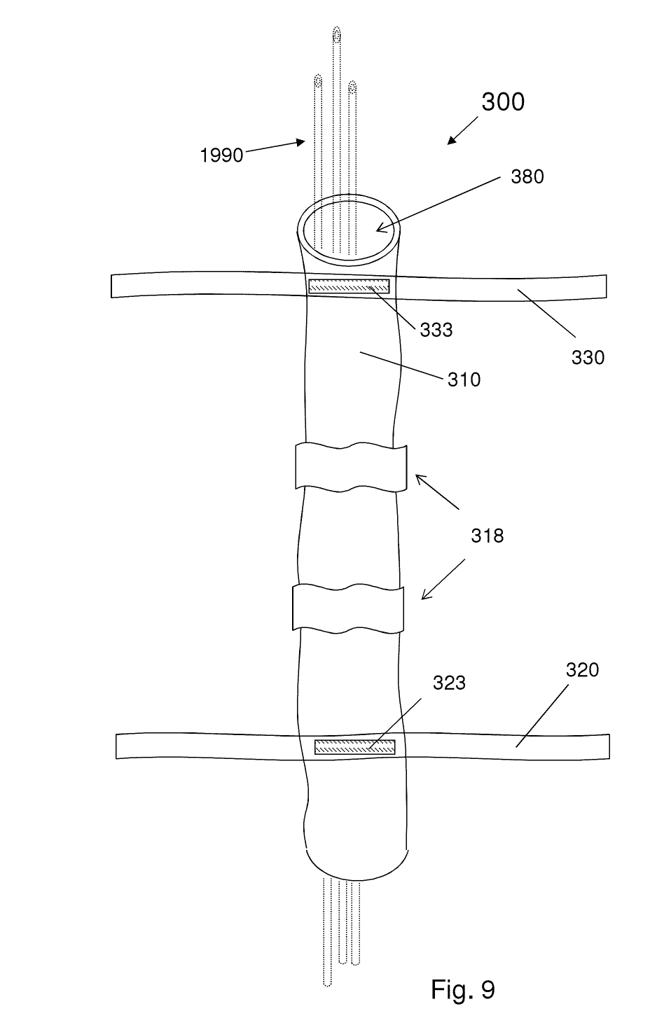

FIG. 9 is a side view of a medical conduit protection device 300 in accordance with a third embodiment of the invention, wherein a relatively large diameter body 310 is provided, in conjunction with one more closures 318 arranged circumferentially about the body 310 at locations along the axis of the body 310. As with the foregoing embodiments, a first tether 320 and a second tether 330 are provided, each being attached to the body 310 in respective areas 323, 333. The body 310 is provided with a relatively large diameter lumen 380 and is, in one preferred embodiment, formed from a relatively compliant material, but preferably one that does not stretch significantly such as a nonwoven material such as Tyvek®, for example. It is particularly conceived that use of such a material advantageously provides all of the benefits of the invention at a relatively low manufacturing cost. If constructed relatively simply from such a material, the tethers 320, 330 can also be made from such material and the whole device 300 can be made to be recyclable, among other advantages.

As illustrated, the medical conduit protection device 300 does not include a longitudinal opening, but only first and second and openings to access the lumen 380. However, it is to be understood that a longitudinal closure can be applied in conjunction with features described in connection with this embodiment.

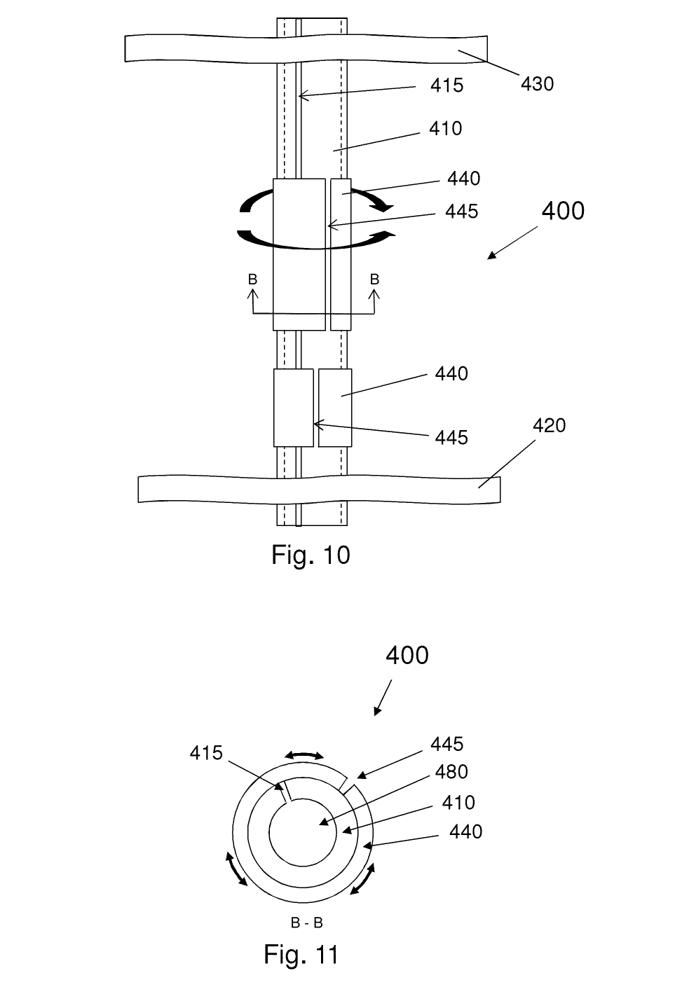

FIGS. 10-11 illustrate a medical conduit protection device 400 in accordance with a fourth embodiment of the invention, wherein rotatable closure elements 440 are provided for inhibiting access to conduits held within a lumen 480 of the device 400. As with foregoing embodiments, first and second tethers 420, 430 are provided at respective ends of the body 410. As can be seen best in the cross-sectional view of FIG. 11, the body 410 is provided with a longitudinal slit 415 to permit transverse insertion of medical conduits into the lumen 480. In this case, two rotating closure elements 440 are provided concentrically around the body 410. The rotating closure elements 440 have respective slits 445 formed therein. When the slit 445 aligns with the slit 415, medical conduits can be inserted into or removed from the lumen 480. When the rotating closure elements 440 the are rotated about the longitudinal axis of the body 410, the slit 445 no longer aligns with the slit 415, and access to the lumen 480 is inhibited. If so desired, one or more rotating closure elements 440 can be provided such that they cover all or substantially all of the length of the body 410, thereby further inhibiting axis to the lumen 480.

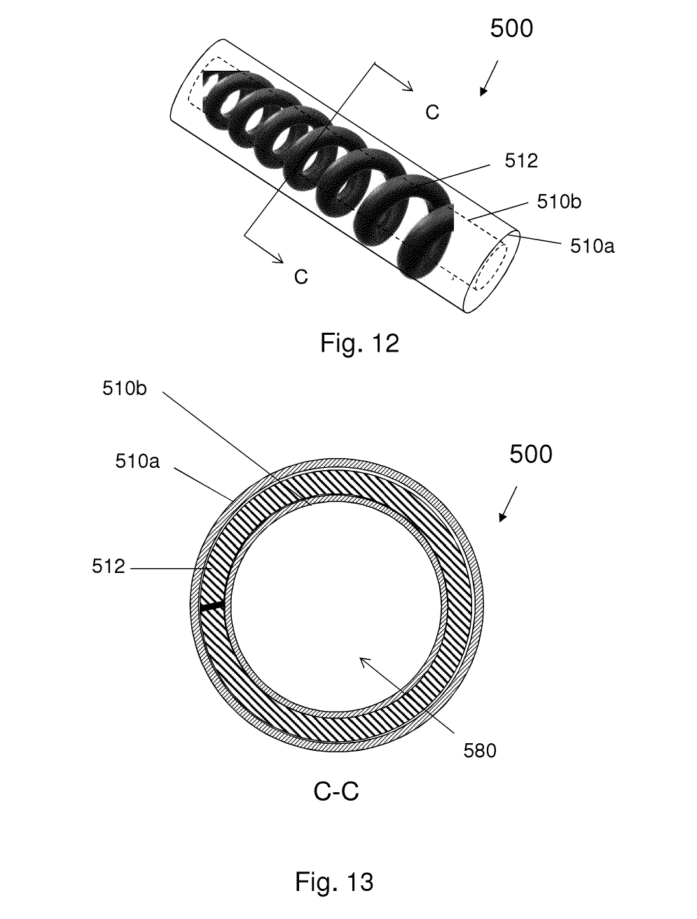

FIGS. 12 and 13 illustrate a medical conduit protection device 500 in accordance with a fifth embodiment of the invention, wherein a helical structural element 512 is provided in connection with the body 510 of the device. Such an arrangement advantageously imparts structure to the device 500 while also physically shielding contents of the lumen 580 from external influences. Additionally, the device 500 is longitudinally compressible such that the device takes up minimal space in storage and during transportation. In the illustrated embodiment, as best seen in the cross-sectional view of FIG. 13, the body can include an inner layer of material 510b as well as an outer layer of material 510a. The structural element 512 can be formed of any suitable material, including, but not limited to metals or semi-rigid polymeric materials.

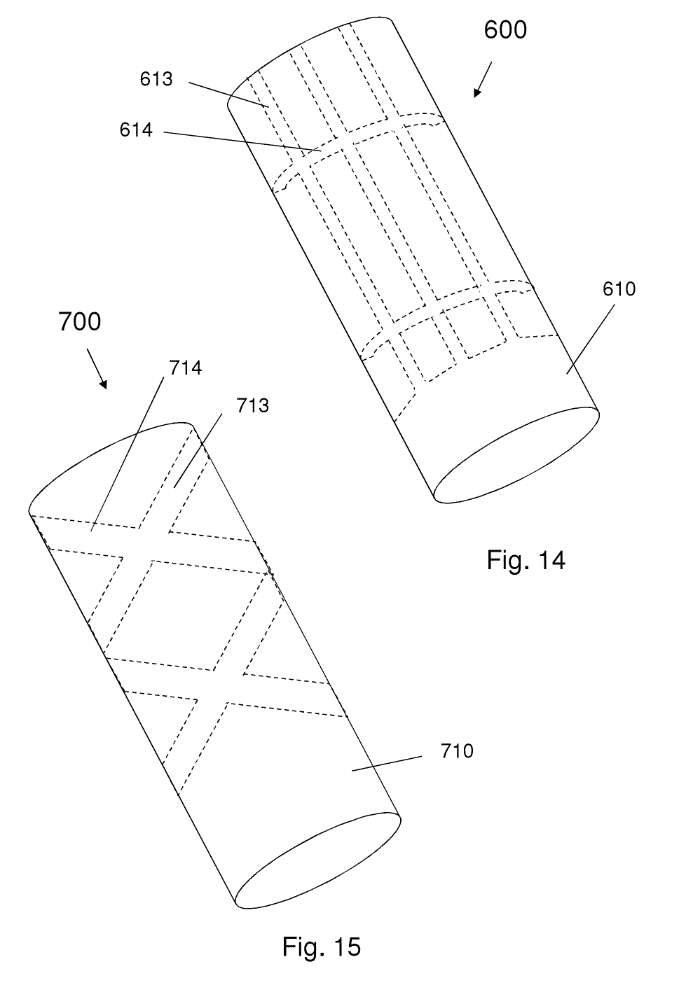

FIG. 14 is an end isometric view of a medical conduit protection device 600 in accordance with a sixth embodiment of the invention, wherein inflatable channels 613, 614 are provided in connection with the body 610 of the device 600 in a longitudinal and circumferential pattern to enhance structural rigidity of the body 610, and to physically shield the contents of the lumen from tampering.

FIG. 15 illustrates a medical conduit protection device 700 in accordance with a seventh embodiment of the invention, wherein inflatable channels 713, 714 are provided in connection with the body 710 of the device 700 in a pattern of dual oppositely winding helices about the circumference of the body 710 (simply, “diagonal” channels), to enhance structural rigidity of the body 710.

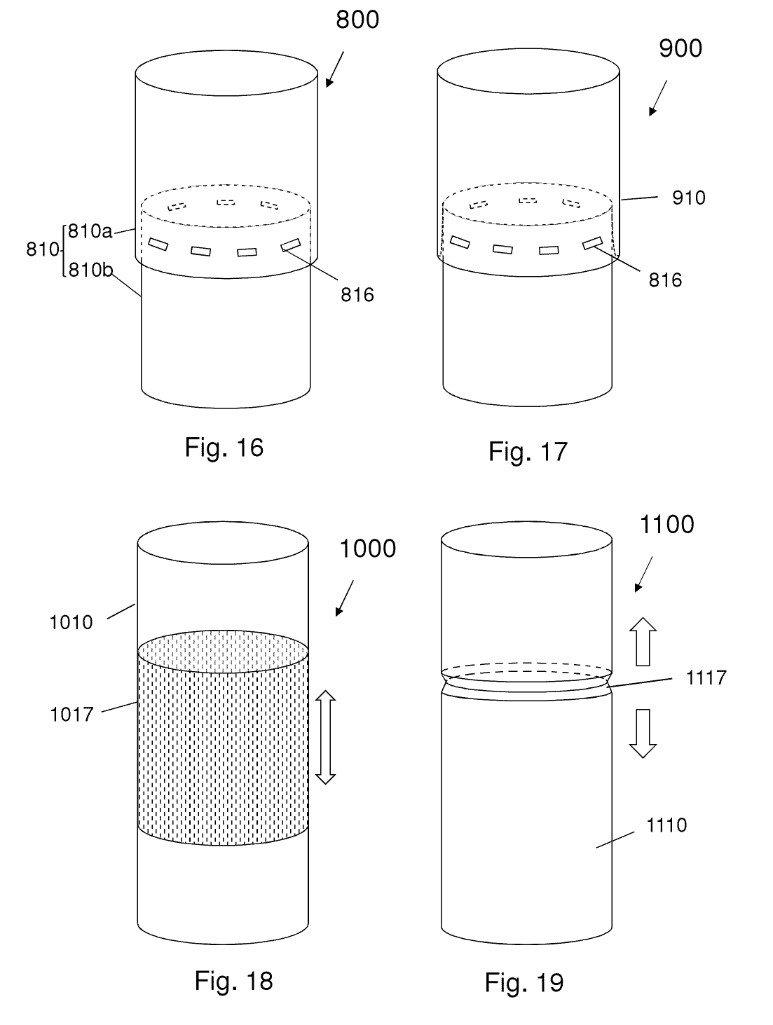

FIG. 16 illustrates a medical conduit protection device 800 in accordance with an eighth embodiment of the invention, which is provided with a safety release feature, in which connections between two segments 810a, 810b of a body 810 are connected by severable or otherwise releasable connections 816. Such releasable connections 816 can be, for example, stitches or staples the that release upon application of a predetermined longitudinal force to the body 810, based on the material properties that are selected.

Similarly, FIG. 17 illustrates a medical conduit protection device 900 in accordance with a ninth embodiment of the invention, which is provided with a safety release feature, in which connections between two segments of a continuous body 910 are folded over one another and connected by severable or otherwise releasable connections 816. While the two segments 810a, 810b of the body 810 of the device illustrated in FIG. 16 would separate from one another upon application of sufficient force, the overlapped section of the device 900 illustrated in FIG. 17 would separate, the body 910 would remain contiguous.

FIG. 18 illustrates a medical conduit protection device 1000 in accordance with a tenth embodiment of the invention, which is provided with a safety feature, in which a segment 1017 of the body 1010 is formed of a resilient material, such as an elastomeric material for example, to permit limited stretching of the body 1010 of the device 1000.

FIG. 19 illustrates a medical conduit protection device 1100 in accordance with an eleventh embodiment of the invention, which is provided with a safety release feature, in which a line of weakness 1117 is provided in the body 1110. As embodied, if a longitudinal force exceeding a predetermined value is applied to the body 1110, the body 1110 will first separate along the line of weakness 1117. Advantageously, an alarm feature can be integrated with such a line of weakness so that the alarm is triggered if excessive force is applied to the body 1100, such as a severable conductive element spanning between the two segments, for example.

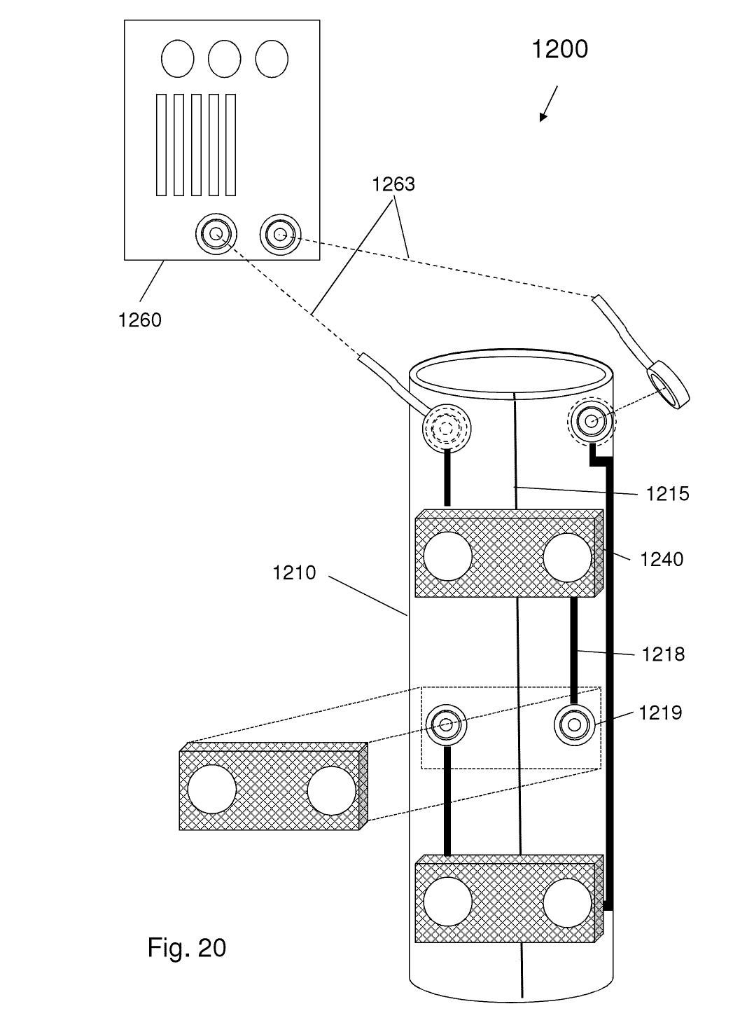

FIG. 20 illustrates a medical conduit protection device 1200 in accordance with a twelfth embodiment of the invention, which is provided with an alarm 1260 to dissuade tampering with the device 1200. The device 1200 includes a body 1210 having a longitudinal slit 1215. A plurality of closure elements 1240 are provided which, in the illustrated embodiment, connect to the body 1210 by way of conductive (e.g. metal) snaps. Accordingly, various conductive elements 1218 provided on the body 1210 can together complete an electrical circuit with electrical connections 1263 and the alarm 1260. In accordance with the illustrated embodiment, the closure elements 1240 can be made of any suitable material as long as they are provided with a conductive path, such as a wire, to complete the electrical circuit illustrated. Although other circuit arrangements can be implemented, in the case of a series of arrangement such as that illustrated, a break in any one of the connections would cause the alarm to sound.

If so desired, the closure elements 1240 can be formed from something as simple as the metallized sheet connected to the body 1210 by way of conductive elements which can be, if desired, one-way connectors that do not permit removal without destruction of at least a portion thereof, thus enhancing tamper resistance and tamper evidence of the subject devices.

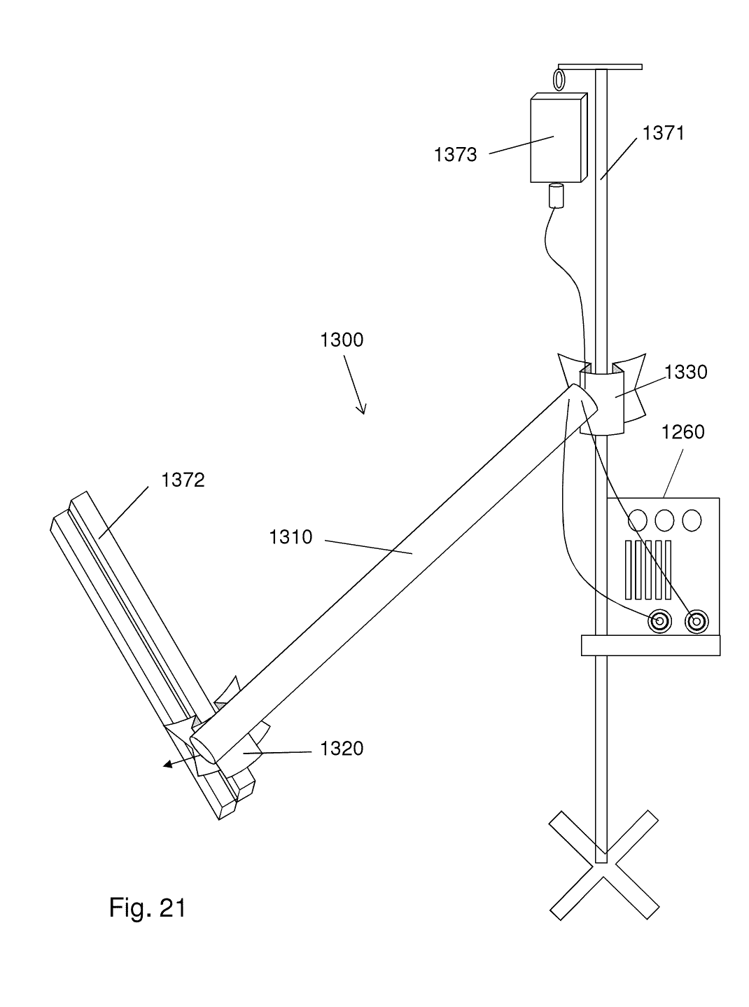

FIG. 21 illustrates a medical conduit protection device 1300, in use, in accordance with the invention, secured between an IV pole 1371 and a bed rail 1372. The device 1300 includes a body 1310 and tethers 1320, 1330 which are respectively secured to the bed rail 1372 and the IV pole 1371. Also illustrated is an IV pouch 1373, from which a line extends into and through a lumen of the body 1310.

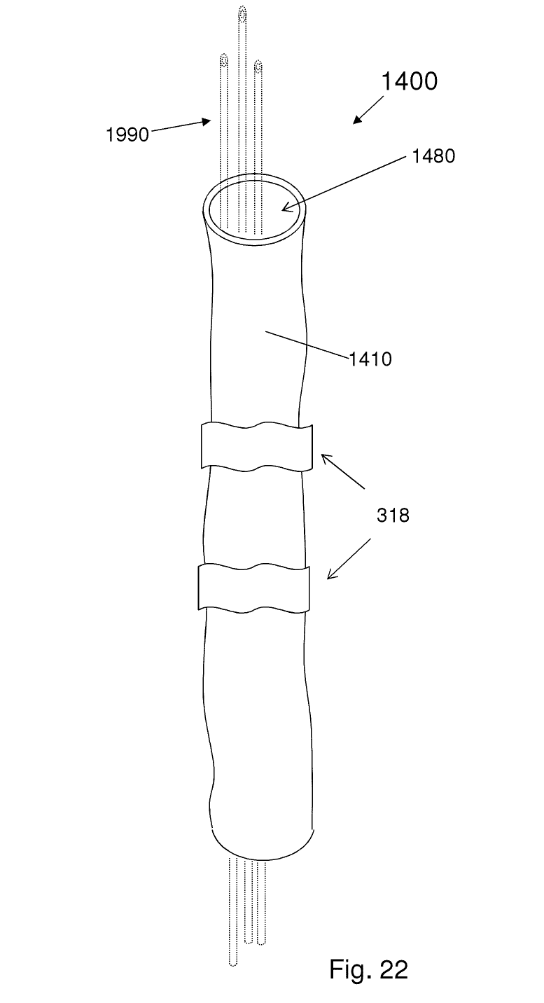

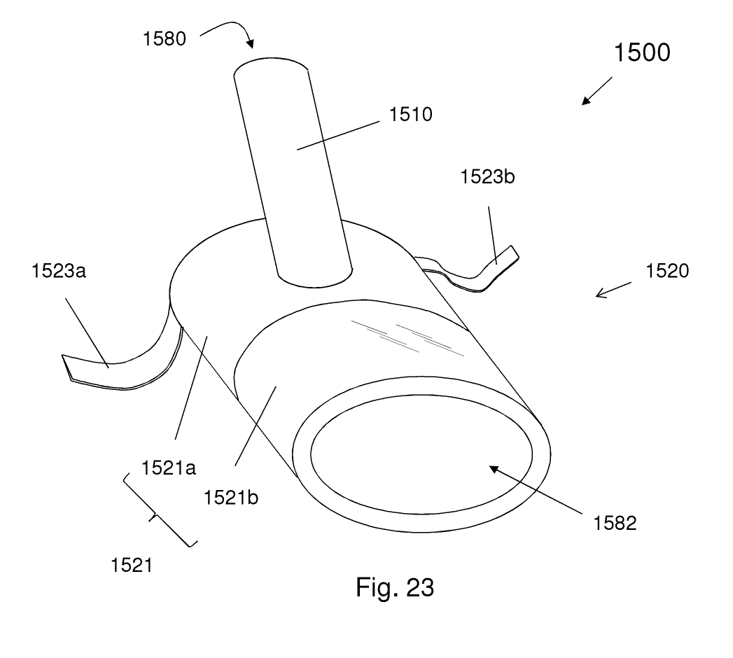

FIG. 22 is a side view of a medical conduit protection device 1400 in accordance with a thirteenth embodiment of the invention, wherein a relatively large diameter body 1410 is provided, in conjunction with one more closures 318 arranged circumferentially about the body 1410 at locations along the axis of the body 1410, similarly to the embodiment of FIG. 9.

Unlike the foregoing embodiments, neither a first tether nor a second tether are provided. Instead, a patient end of the protection device 1400 may be attached directly to the patient as needed, such as by medical tape or other dressing. Further, a far end can simply be draped over the conduits to be protected, and remain in place, either by the inherent structural properties of the device, by friction, by application of a separate tie or restraint, or other force, depending on the precise implementation. Further, it is to be understood that only one of the end tethers can be eliminated, if desired. In such embodiments, the subject protection devices will not distribute applied external forces as fully as with the foregoing embodiments, but tampering with or damage to the attached conduits, and other advantages, can still be achieved.

FIG. 23 is an isometric view of a medical conduit protection device 1500 in accordance with a thirteenth embodiment of the invention, in which a lumen 1580 of the body 1510 of the device is contiguous with a lumen 1582 formed in a cuff 1521 of a patient-end tether 1520 thereof. Such a configuration permits passage of and protection of patient-connected medical conduits within the medical conduit protection device 1500 up to and including protection of the site on a patient where such medical conduits are attached to or inserted into the patient's body.

Furthermore, as illustrated, although optional, a portion 1521b of the cuff 1521 is made from a transparent material to permit monitoring of an insertion site of patient-connected medical conduits. As such, a healthcare professional is able to more easily monitor the insertion site for any movement of conduits, bleeding, infection or infiltration of any medicament into surrounding tissues.

As illustrated, ties 1523a, 1523b are optionally provided on the non-transparent part 1521a of the cuff 1521 so as to facilitate attachment to the patient. However, other securing features can be provided in place of or in addition to such ties 1523a, 1523b.

In accordance with the invention many from materials can be utilized in the manufacture of the subject devices. Materials can include but are not limited to woven fabrics, nonwoven fabrics, plastics, metallized plastics, elastomers, laminated sheets, composite sheets, rigid tubes, semi-rigid tubes and others. The subject devices can be fabricated by way of any suitable technique including, but not limited to, heat welding, solvent welding, stitching, adhesives, staples and the like.

If so desired, magnetic elements can be provided for closure of the body openings and/or for connection of the subject devices to external points such as an IV pole. Further, if desired, one or more strain gauges can be applied to the bodies of the subject devices or to the tethers, for example. Additionally or alternatively, if desired, the subject devices can be provided with further features for safety including, but not limited to, an integrated panic button electrically connected to the alarm and/or a releasable ripcord to quickly open up the subject devices, should a problem arise needing rapid attention, for example.

The alarm features of the subject devices can include more sophisticated detection circuitry than simply requiring completion of a series circuit to maintain an alarm in the off state. Such circuits can include measurement of minute changes in resistance, changes in capacitance of various elements that may be provided on such devices, and/or monitoring the status of one or more strain gauges integral to the subject devices, for example. Further, location information based on detection of proximity sensors in a hospital room, on the floor of a ward, or elsewhere in a building can be incorporated, for example, to permit higher variance of detected impedance when walking or otherwise moving. Conversely, if the patient is in bed, the alarm circuitry can be configured so as to have reduced tolerance of detected disturbances (such as a duration and/or magnitude of changes in parameters such as capacitance or resistance). In this way, it may be possible to accurately differentiate between a patient who is simply moving around to go to the bathroom and a patient who is actively tampering with the subject devices.

Further, if an alarm feature is provided in connection with devices in accordance with the invention that have one or more inflatable portions, one or more pressure gauges can be utilized to detect a fluctuation or larger changes in pressure—higher or lower than an initial pressure—in the chambers of such devices which may indicate tampering.

While the devices and related methods of subject invention have been shown and described with reference to selected embodiments, those skilled in the art will readily appreciate that changes and/or modifications may be made thereto without departing from the spirit and scope of the subject invention. Moreover, it should be understood that elements or features described in connection with any embodiment disclosed herein can be advantageously applied to other embodiments described herein, if practicable and not mutually exclusive with other features of such embodiments.

What is claimed is:

1. A medical conduit protection device comprising:
a.) an elongate body having a wall, defining a lumen therein, and having first and second opposed ends, the body being adapted and configured to receive, within the lumen thereof, one or more elongate medical conduits;
b.) a first tether provided on the first end of the body, the first tether being adapted and configured to secure the first end of the body to a patient-end anchor point; and
c.) a second tether provided on the second end of the body, the second tether being adapted and configured to secure the second end of the body to an external anchor point,
d.) the medical conduit protection device inhibiting tampering with or damage to the one or more medical conduits within the lumen of the body by transferring externally-applied strain away from the one or more medical conduits within the lumen thereof.

2. The medical conduit protection device of claim 1, wherein the body is a substantially continuous tubular structure, adapted and configured to receive one or more medical conduits through the lumen thereof.

3. The medical conduit protection device of claim 2, wherein the body is formed from a resilient material which is resistant to plastic deformation.

4. The medical conduit protection device of claim 2, wherein the body is formed from a nonwoven fabric.

5. The medical conduit protection device of claim 2, wherein the body has a diameter of between about 2.0 centimeters and about 5.0 centimeters.

6. The medical conduit protection device of claim 1, further comprising:
one or more closure elements adapted and configured for inhibiting access to the one or more medical conduits within the lumen.

7. The medical conduit protection device of claim 1, further comprising:
at least one closure element provided in connection with the body, for inhibiting access to the lumen.

8. The medical conduit protection device of claim 7, wherein the at least one closure element is a reversibly attached closure element.

9. The medical conduit protection device of claim 7, wherein the at least one closure element is adapted and configured to require a separate tool for removal thereof.

10. The medical conduit protection device of claim 7, wherein the at least one closure element is a one-way closure element.

11. The medical conduit protection device of claim 7, wherein the at least one closure element is a concentrically rotatable tubular element arranged over the body.

12. The medical conduit protection device of claim 1, wherein the body includes at least one inflatable element, the at least one inflatable element being adapted and configured to impart structural rigidity to at least a portion of the body.

13. The medical conduit protection device of claim 1, wherein the body includes at least one semi-rigid element, the at least one semi-rigid element being adapted and configured to impart structural rigidity to at least a portion of the body.

14. The medical conduit protection device of claim 13, wherein the at least one semi-rigid element is helical spine provided in connection with the wall of the body.

15. The medical conduit protection device of claim 1, further comprising:
an alarm adapted and configured to sound when detecting tampering with the medical conduit protection device.

16. The medical conduit protection device of claim 1, further comprising:
a safety release feature, adapted and configured to release when a force exceeding a predetermined value is applied to the medical conduit protection device.

17. The medical conduit protection device of claim 16, further comprising an alarm provided in connection with the safety release feature, adapted and configured such that when the force exceeding the predetermined value is applied to the medical conduit protection device, the alarm is sounded.

18. The medical conduit protection device of claim 16, wherein the safety release feature includes one or more of a:
line of weakness, severable connection, releasable connection, and elastic elements.

19. A method of protecting medical conduits from damage or tampering, the method comprising:
a) providing a medical conduit protection device, the medical conduit protection device being adapted and configured for attachment at a first end to a patient-end anchor point and at a second end to an external anchor point apart from the patient;
b) arranging the medical conduit protection device about one or more medical conduits so as to inhibit access to the one or more medical conduits, and to distribute externally-applied forces away from the one or more medical conduits;
c) securing the first end of the protection device to the patient;
d) securing the second end of the protection device to an anchor point apart from the patient; and
e) connecting the one or more medical conduits at the first end to the patient-end anchor point and at the second end to medical equipment.

20. A medical conduit protection device comprising:
a.) an elongate body having a wall, defining a lumen therein, and having first and second opposed ends, the body being adapted and configured to receive, within the lumen thereof, one or more elongate medical conduits,
b.) the first end of the body being adapted and configured to be secured directly to a patient-end anchor point,
c.) the medical conduit protection device inhibiting tampering with or damage to the one or more medical conduits within the lumen of the body by shielding from damage the one or more medical conduits within the lumen thereof.
